(19) United States
(12) Patent Application Publication
(10) Pub. No.: US 2020/0113704 A1
(43) Pub. Date: Apr. 16, 2020

(54) CHARCOT TRABECULAR SYSTEM AND METHOD FOR LIMB SALVAGE SURGERY

(71) Applicants: Ryan Pereira, St. Augustine, FL (US); Peter Wayte, St. Augustine, FL (US)

(72) Inventors: Ryan Pereira, St. Augustine, FL (US); Peter Wayte, St. Augustine, FL (US)

(21) Appl. No.: 16/598,637

(22) Filed: Oct. 10, 2019

Related U.S. Application Data
(60) Provisional application No. 62/743,738, filed on Oct. 10, 2018.

(51) Int. Cl. A61F 2/42; A61F 2/30

(52) U.S. Cl. CPC A61F 2/4225; A61F 2/4202; A61F 2/30771; A61F 2310/00023; A61F 2002/30013; A61F 2002/30028; A61F 2310/00131; A61F 2002/3085

(57) ABSTRACT

A Charcot trabecular system and method is provided. The Charcot trabecular system embodies a threaded bolt-like fastener, wherein a middle portion of the shank of the systemic fastener is made of a porous material, while the remaining portions of the systemic fastener denser provides a denser material. The porous shank portion may be made of material dimensioned and adapted to pass blood and particulate bone matter therethrough. As a result, in use when connecting bones together, the denser material provides for stabilization and structural support to the associated damaged joint, while the porous shank facilitates optimal bone healing via boney ingrowth and bone ongrowth through and around the implanted systemic fastener.

### CROSS-REFERENCE TO RELATED APPLICATION

This application claims the benefit of priority of U.S. provisional application No. 62/743,738, filed 10 OCT 2018, the contents of which are herein incorporated by reference.

### BACKGROUND OF THE INVENTION

The present invention relates to medical devices and, more particularly, a Charcot trabecular system for limb salvage. The Charcot trabecular system embodies a joint-repairing implant having a porous body, so that while the load-bearing implant corrects foot and ankle deformities, the porous body promotes both bone ongrowth and bone ingrowth at the repaired joint.

Neuropathic arthropathy, also known as Charcot, is a progressive degeneration of a weight-bearing foot or ankle joint. Charcot is marked by boney destruction, bone resorption, joint deformity, ulceration, superinfection, loss of function, and, in acute Charcot, possibly amputation and/or death. Patients with acute Charcot typically only have five years survivorship or require amputation of the leg below the knee.

Current acute Charcot treatments do not allow for bony ingress in the foot and ankle bones for complex limb salvage. They are simply a solid bolt or screw connecting damaged joints. In other words, such load-bearing implants do not promote optimal osseointegration or osteointegration, which is the direct structural and functional connection between living bone and the surface of a load-bearing artificial implant. Osteointegration is marked by both bone ingrowth and bone ongrowth, where bone ingrowth is firm fixation between bone and metal through bone growth into a porous surface of an artificial implant, while bone ongrowth is fixation between metal and bone through bone growth on a rough surface of an artificial implant.

Today's solid connectors for limb salvage do not allow the repaired joint to heal or return to their former condition as their density impedes both bone ingrowth and bone ongrowth. In short, current options use solid connectors which do not permit penetration of blood or bone matter through—bony ingress—the body of the joint-repairing implant with which it has direct contact, thereby limiting optimal bone healing. Furthermore, current solutions do not allow for a complete rebar system with healing through and around the implant.

As can be seen, there is a need for a Charcot trabecular system for limb salvage embodying a joint-repairing implant having a porous body, so that while the load-bearing implant corrects foot and ankle deformities, the porous body promotes both bone ongrowth and bone ingrowth at the repaired joint. The present invention allows for healing through and around the implant by having the connector having, at least in part, a strong, porous sponge-like material along its body. The sponge-like connector may be attached at either or both ends to denser material having the strength for penetrating bone tissue during insertion as well as for providing stability and strength to associated joints. The dense material strengthens the associated joints while the body regenerates, wherein ingress of blood and particulate bone matter in and through the porous sponge-like material engaging the damaged joint encourages repair of the joint by way of bone ingrowth and bone ongrowth, thereby enabling patients to have a normal life expectancy.

The charcot trabecular system may be used for complex foot and ankle reconstructive surgical procedures and allows optimal stability and strength of the repaired joints while the damaged bone heals via osseous ingress to reform the boney anatomy while in its stabilized configuration.

### SUMMARY OF THE INVENTION

In one aspect of the present invention, a Charcot trabecular systemic implant includes the following: a fastener extending from a head end to a tip end; a porous shank interconnecting the head end and the tip end; the porous shank made from a porous material dimensioned to pass blood and particulate bone matter therethrough; and the head and tip ends made from a dense material denser than the porous shank, wherein the dense material is titanium, tantalum, or a combination thereof; and a tip circumferential threading disposed between the porous shank and a distal end of the tip end; and a head circumferential threading disposed between the porous shank and a distal end of the head end.

In another aspect of the present invention, a method for promoting bony ingress in the foot and ankle bones during limb salvage surgery includes the following: implanting the above-mentioned Charcot trabecular systemic implant into one or more foot, leg, and/or ankle bones of a patient so as to provide load-bearing stability to associated joints, wherein the Charcot trabecular systemic implant is generally coplanar with at least one of the one or more foot, leg, and/or ankle bones so as to corrects foot and ankle deformities, and wherein the porous shank is generally fully embedded in said at least one of the one or more foot, leg, and/or ankle bones.

These and other features, aspects and advantages of the present invention will become better understood with reference to the following drawings, description and claims.

### BRIEF DESCRIPTION OF THE DRAWINGS

FIG. 1 is a side elevation view of an exemplary embodiment of the present invention;

FIG. 2 is a side elevation view of an exemplary embodiment of the present invention, shown in use;

FIG. 3 is a cross-sectional view of an exemplary embodiment of the present invention, taken along line 3-3 in FIG. 2; and

FIG. 4 is a side elevation view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a Charcot trabecular system embodying a threaded bolt-like fastener, wherein a middle portion of the shank of the systemic fastener is made of a porous material, while the remaining portions of the systemic fastener denser provides a denser material. The porous shank portion may be made of material dimensioned and adapted to pass blood and particulate bone matter therethrough. As a result, in use when connecting bones together, the denser material provides for stabilization and structural support to the associated damaged joint, while the porous shank facilitates optimal bone healing via boney ingrowth and bone ongrowth through and around the implanted systemic fastener.

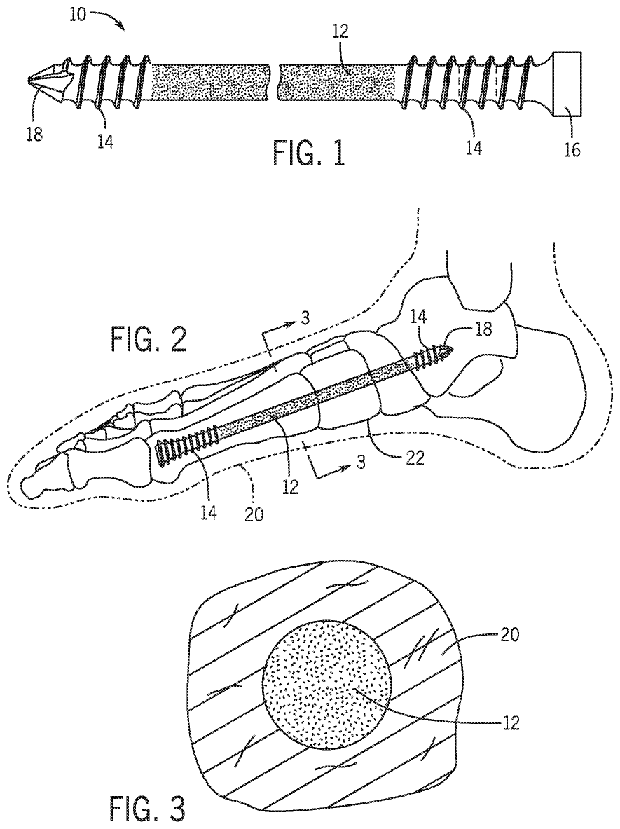

Referring now to FIG. 1, the present invention may include a Charcot trabecular system embodying a threaded bolt-like partially porous fastener 10. The systemic fastener 10 may extend from a head end 16 to a tip end 18. Disposed between the tip end 18 and the head end 16 may be porous shank 12. Between the porous shank 12 and the head end 16 and the tip end 18 may be threaded shanks 14. The systemic fastener 10, except for the porous shank 12, may be made of Titanium Ti64, Tantalum, a combination thereof, or a suitably strong and dense material or alloy compatible with human blood and tissue. The porous shank 12 may be made of a strong sponge-like porous material capable of withstanding the stresses incurred by repaired joints during use while allowing blood and particulate bone matter to pass there through.

The present invention may be manufactured using integrated computational adaptive additive manufacturing (iCAAM) process, a software-based process designed to manufacture high integrity parts. Through this manufacturing process the Ti64 or Tantalum may be deposited in a solid, dense condition at either or both ends of the sponge configured porous shank 12. The portion of the denser material may be threaded adjacent to the porous shank 12 to allow connection into bone during implantation.

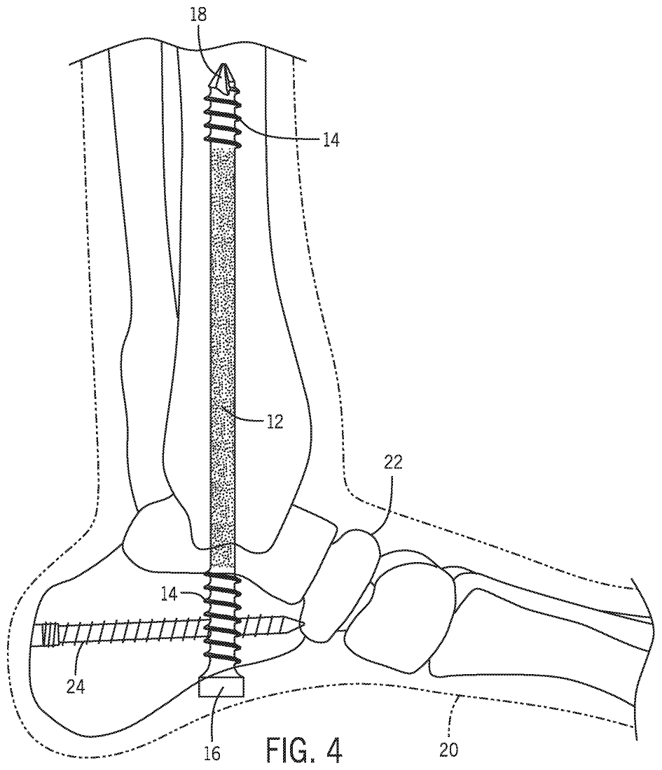

Referring to FIGS. 2 through 4, a method of using the present invention may include the following. The systemic fastener 10 disclosed above may be provided. A surgeon during complex foot and ankle reconstruction may implant the systemic fastener 10 into a foot, leg, and/or ankle bone 22 of a patient 20, optimizing bone healing via bony ingrowth and ongrowth through and around the implanted systemic fastener 10, which also provides strength and support to a damaged joint. The systemic fastener 10 may be used in conjunction with pre-existing bone and joint strengthening and stabilizing fasteners 24, such as screws.

It should be understood, of course, that the foregoing relates to exemplary embodiments of the invention and that modifications may be made without departing from the spirit and scope of the invention as set forth in the following claims.

What is claimed is:

1. A Charcot trabecular systemic implant, comprising:
a fastener extending from a head end to a tip end;
a porous shank interconnecting the head end and the tip end;
the porous shank made from a porous material dimensioned to pass blood and particulate bone matter therethrough; and
the head and tip ends made from a dense material denser than the porous shank.

2. The Charcot trabecular systemic implant of claim 1, wherein the dense material is titanium, tantalum, or a combination thereof.

3. The Charcot trabecular systemic implant of claim 2, further comprising:
a tip circumferential threading disposed between the porous shank and a distal end of the tip end; and
a head circumferential threading disposed between the porous shank and a distal end of the head end.

4. A method for promoting bony ingress in the foot and ankle bones during limb salvage surgery, comprising:
providing the Charcot trabecular systemic implant of claim 1; and
implanting the Charcot trabecular systemic implant into one or more foot, leg, and/or ankle bones of a patient so as to provide load-bearing stability to associated joints.

5. The method of claim 4, wherein the Charcot trabecular systemic implant is generally coplanar with at least one of the one or more foot, leg, and/or ankle bones so as to corrects foot and ankle deformities.

6. The method of claim 5, wherein the porous shank is generally fully embedded in said at least one of the one or more foot, leg, and/or ankle bones.
